(19) United States
(12) Patent Application Publication
(10) Pub. No.: US 2008/0140002 A1
(43) Pub. Date: Jun. 12, 2008

(54) SYSTEM FOR DELIVERY OF BIOLOGICALLY ACTIVE SUBSTANCES WITH ACTUATING THREE DIMENSIONAL SURFACE

(76) Inventors: Kamal Ramzipoor, Fremont, CA (US); Ary Chernomorsky, Walnut Creek, CA (US)

Correspondence Address: LEVINE BAGADE HAN LLP, 2483 EAST BAYSHORE ROAD, SUITE 100, PALO ALTO, CA, 94303, US

(21) Appl. No.: 11/852,711

(22) Filed: Sep. 10, 2007

Related U.S. Application Data
(60) Provisional application No. 60/868,915, filed on Dec. 6, 2006.

(51) Int. Cl. A61M 25/10

(52) U.S. Cl. 604/103.02

(57) ABSTRACT

Tissue expanding and drug delivery systems with actuating three-dimensional surfaces are described for controlling the delivery and release of therapeutic agents against or upon tissue regions of interest. Such treatments devices and methods may include systems utilizing pores having various pore architectures to control the release of one or more drugs from an outer layer of an expandable delivery instrument, such as a balloon.

### CROSS-REFERENCE TO RELATED APPLICATIONS

The application claims the benefit of priority to U.S. Prov. Pat. App. 60/868,915 filed Dec. 6, 2006, which is incorporated herein by reference in its entirety.

### FIELD OF THE INVENTION

The present invention relates to tissue expanding devices and methods that are removably placed upon a tissue region of interest in a human body to create an opening. The devices may have an actuating surface for delivery of various therapeutic agents into or upon the targeted site.

### BACKGROUND OF THE INVENTION

One of the most common techniques for treatment of vascular occlusive disease is called percutaneous balloon angioplasty or PTA. However, the PTA has a significant drawback that is the high potential for the stenotic vessel to re-close after the procedures, in 30% to 45% of the patients treated, a phenomenon known as re-stenosis. Hence, scaffolds called stents or stent grafts have been developed that stay in place to keep the vessel patent after dilatation. Despite this evolution, stenting is only able to decrease the re-stenosis rate down to 20% to 30% although with additional cost and clinical risks. Advances in drug eluding stents have significantly improved these outcomes by achieving further reduction of re-stenosis rates to the levels of 9%. Unfortunately, this has been eclipsed by reports of complications such as Late Stent Thrombosis, where the blood-clotting inside the stent can occur one or more year's post-stent implantation. While this has been seen rarely in currently marketed devices, thrombosis is extremely dangerous and potentially fatal in over 45% of the cases.

Late Stent Thrombosis usually occurs before endothelialization has been completed. For bare-metal stents, this process takes a few weeks. The drug-eluting stents inhibit re-stenosis by inhibiting fibroblast, proliferation, but they also tend to delay the endothelialization process. Additionally the stents are covered with drug carrier polymers that themselves are often inflammatory to the tissue. Combinations of these two factors may cause a late or incomplete healing of the vessel wall leading to Late Stent Thrombosis.

A local drug delivery device which would deliver predetermined volume and concentration of drugs to the target while avoiding complications associated with the drug-eluting stents would be highly advantageous.

In fact there are several local drug delivery devices, including catheters with permeable balloon membranes and/or perfusion holes to aid with this delivery. However, most are plagued with the rather uniform problem of low transfer efficiency, rapid washout, poor retention, systemic toxicity and the potential for additional vessel injury.

Accordingly, there exists a need for methods and apparatus for effectively and efficiently delivering pharmaceutical agents to a specific location within the blood vessels of a human body.

### SUMMARY OF THE INVENTION

Endovascular treatment of a stenotic lesion may be accomplished by a device that can expand the vessel via a balloon and deliver a therapy such as anti-restenotic and/or anti-thrombosis agents/drugs into the vessel wall. One variation may include a device that contains a balloon with a three-dimensional surface and significant capacity to deliver therapeutic agents/drugs into the vessel.

Such a device may also selectively deliver pharmaceutical agents at predetermined balloon diameters. Since the drug may be released at a given balloon diameter, infusion and washout during delivery and inflation periods may be eliminated, providing for a highly efficient and precise delivery mechanism. Moreover, often times it is desirable to have different agents to address different aspects of the stenotic lesion within the vessel, thus to the device may also be configured to provide for release of a first agent when the balloon reaches its first diameter and the second and third agents (or more), as necessary, when the balloon diameter increases. This is highly beneficial, for example, when encountering thrombosed and stenotic lesions where a device containing fibrolytic and anti restenotic agents can be used. Since presence of the thrombus causes reduction in vessel diameter, the fibrolytic agent may be first released when balloon researches its small diameter, dissolving the thrombus. The balloon may be then fully inflated, releasing the anti-restenotic agent into the vessel wall.

Another embodiment of the device is related to the release of different drugs or different concentrations of the same drug at a given balloon diameter. One example of the use of this feature is addressing edge effect restenosis. Current generation of drug eluting stents have problems with edge effect or restenosis beyond the edges of the stent and progressing around the stent into the interior luminal space.

The causes of edge effect restenosis in first generation drug delivery stents are currently not well understood. It may be that the region of tissue injury due to angioplasty and/or stent implantation extends beyond the diffusion range of current generation agents such as Paclitaxel or Rapamycin, which tend to partition strongly in tissue. Placing higher doses or higher concentrations of agents along the edges, placing different agents at the edges which diffuse more readily through the tissue, or placing different agents or combination of agents at the edges of the treated area may help to remedy the edge effect restenosis problem.

Another example of treatment may include treating a patients having thrombosed vessels, wherein the device is progressively expanded to various diameters, each time releasing a dose of fibrolytic agent dissolving thrombosis immediately surrounding the balloon until the entire lumen is cleared and a full recanalization is achieved.

### BRIEF DESCRIPTION OF THE DRAWINGS

FIG. 1A shows an illustrative view of vessel of a patient body with a variation of the treatment system minimum invasively positioned therein.

FIG. 1B shows a partial cross-sectional detail view of a variation of the catheter apparatus having a balloon with a surface for expanding and temporarily contacting and delivering pharmaceutical agents/drugs into the vessel wall.

FIG. 2A shows a partial cross-sectional view of the catheter apparatus placed within a vessel

FIG. 2B shows a partial cross-sectional view of the catheter with a balloon having a surface and at least partially expanded within a vessel.

FIG. 2C shows a partial cross-sectional view of the balloon having an absorbent surface and fully expanded and apposed against the interior of the vessel releasing agents/drugs into the vessel wall.

FIG. 3 illustrates release of different agents or different concentrations of the same agent at locations distal and proximal to the balloon to address such disorders as “edge effect restenosis”.

FIG. 4A illustrates a catheter apparatus with a balloon having a surface with longitudinal segments capable of un-compressing when the balloon diameter is relatively small.

FIG. 4B illustrates a catheter apparatus with a balloon partially expanded, releasing fibrolytic agents.

FIG. 4C illustrates a catheter apparatus with a balloon fully expanded, releasing anti-restenotic agents.

FIGS. 5A to 5C show a cross-section view of the drug delivery balloon with outer porous layer going through the inflation process with the consequent changes in the pore architecture and dimensions.

FIG. 5D shows an enlarged cross sectional view of the balloon segment having an outer layer containing predetermined pore architecture.

FIGS. 6A and 6B show longitudinal views of the enlarged segments of a porous layer having another predetermined pore architecture.

FIGS. 6C and 6D show enlarged segments of a porous layer that includes a plurality of porous fibers having yet another predetermined pore architecture.

FIG. 7A illustrates an example of the stacked structure of a porous layer.

FIG. 7B illustrates another example of yet another stacked structure with different pore architecture and orientation of a porous layer.

FIG. 8A is a photomicrograph of a porous layer having a predetermined pore architecture.

FIG. 8B is another photomicrograph of a porous layer having a predetermined pore architecture.

FIG. 8C is another photomicrograph of a porous layer having a predetermined pore architecture.

FIG. 8D is yet another photomicrograph of a porous layer having another predetermined pore architecture.

FIG. 8E is another photomicrograph of having still another predetermined pore architecture.

FIG. 9A is a combination of photomicrographs of porous layers illustrating the formation of a stacked laminate structure including a first layer having a first predetermined pore architecture and a second layer having a second predetermined pore structure.

FIG. 9B is a combination of photomicrographs of a porous layer that collectively illustrate a predetermined pore density gradient and/or predetermined size gradient.

FIGS. 10A to 10C illustrate delivery and release of a stent in combination with infusion of a therapeutic agent into the targeted site.

FIG. 11A is a perspective view of the three-dimensional substrate sleeve.

FIG. 11B is a perspective view of the substrate sleeve placed on the catheter balloon which shows the three-dimensional porous nature of the substrate.

FIG. 11C is a longitudinal view of the substrate sleeve fitted on the balloon

FIG. 11D is an enlarged longitudinal view of the substrate sleeve fitted on the balloon in its inflated state and shows the configuration of the pores throughout the thickness of the substrate wall.

FIG. 12A shows the substrate sleeve covered with a polymeric film.

FIG. 12B illustrates the expansion of the balloon and as a consequence of that disintegration and defragmentation of the coating film turning it into a disintegrated surface.

FIG. 12C shows further disintegration of the coating film into even smaller fragment which are either soluble or degradable by the physiological environment.

FIG. 12D show a fully inflated balloon, covered with a substrate sleeve completely free of coating.

FIGS. 13A to 13C illustrate additional variations of the expandable balloon covered with a sleeve which have various configurations for reservoirs along the sleeve surface which are capable of expanding when the balloon reaches a predetermined diameter to release any biologically active substances.

FIG. 13D illustrates a cross-sectional end view of the balloon having an outer layer and an example of reservoir architecture.

FIGS. 14A and 14B show perspective and cross-sectional end views, respectively, of another variation for reservoir configuration.

FIG. 15 is a graph showing an increase in pore size and correlated release of a drug agent when the balloon reaches its maximum diameter.

FIG. 16 is a graph showing the maximum release of a drag agent at a predetermined balloon diameter of, e.g., 4 mm.

FIG. 17 is a graph showing an example of two different pore architectures responding to the balloon expansion.

FIG. 18 is a graph showing 100% release of a first drug agent when balloon researches its first diameter of, e.g., 3 mm, followed by complete release of a second drug agent when the balloon is fully inflated to, e.g., 4 mm diameter.

FIGS. 19A to 19D show cross-sectional views of the drug delivery balloon with outer porous layer covered with outer sheath with structurally jeopardized surface, going through the inflation process with the consequent changes in the pore architecture and dimensions and the outer sheath that disintegrates under radial stresses generated during inflation of the balloon.

FIGS. 20A and 20B illustrate an outer sheath with structurally jeopardized surface were longitudinal cut are pre made to accelerate a peel-off process.

FIGS. 21A and 21B illustrate an outer sheath with a structurally jeopardized surface having multiple perforations or holes to allow elution of the biological agent under pressure.

FIGS. 22A to 22D illustrate an outer sleeve made from a thin layer of biodegradable material with a mechanically jeopardized surface having multiple cuts and/or holes to accelerate the process of bioabsorption under pressure to allow elution of the biological agent.

FIGS. 23A to 23D illustrate an outer sleeve made out of a thin, layer of material which is degradable under application of energy.

### DETAILED DESCRIPTION OF THE INVENTION

Although devices and methods are described relative to a biologically active substance applied to the Interior of the blood vessel device, it is to be understood that the other variations are not to be limited thereby. Indeed, other variations may be advantageously utilized for simultaneous angioplasty and anti-restenosis treatment of various blood vessels.

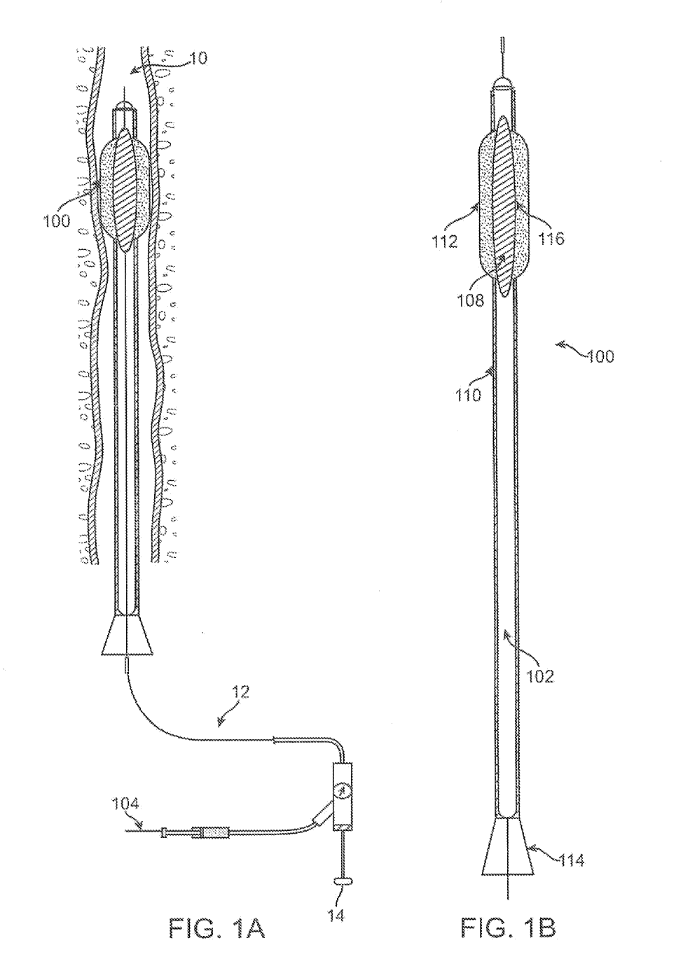

FIG. 1A illustrates an illustrative view of a blood vessel 10 of a patient body with one variation of the catheter treatment system 100 positioned therein. The catheter treatment system 100 may be introduced into the patient body via percutaneous access through the patient's skin and into the blood vessel 10 to be treated. The catheter system 100 may be advanced into the blood vessel 10 until the portion to be treated has been reached and/or traversed by the catheter system 100. As further shown, the catheter treatment system 100 may be connected via an inflation/deflation tubular member 12 to a pump 14 positioned externally of the patient.

Moreover, one or more access ports may be incorporated with the system to allow for access by other devices, such as guidewire 104, which may be optionally advanced distally of the catheter system 100 to facilitate access through the blood vessel. Additionally, a proximal portion 114 of the catheter assembly 100 may further define a flared or tapered portion to facilitate the insertion and access of a guidewire 104 into and through the assembly 100.

FIG. 1B illustrates one variation of an elongated tubular catheter assembly 100, having a distal and a proximal end and a lumen 102 to optionally receive a guidewire 104 therethrough. The catheter assembly 100 also includes an inflation balloon 108, and an inflation lumen 110 that is in fluid communication with the balloon 108. The outer surface 116 of the balloon 108 may be completely or at least partially covered with a highly absorbent material such as foam 112 or other absorbent materials, as further described below. The outer surface 116 of the balloon 108 may be comprised of a retaining material to facilitate the absorption and retention of an agent/drug therein. Such a retaining material may include any number of substances which are configured to retain and/or absorb a biological or non-biological liquid or solid medium. Such materials may be accordingly configured to include a number of reservoirs for retaining the liquid or solid medium where reservoirs may include any liquid or solid medium retaining structures, e.g., pores, troughs, capacitors/capacitance (which used herein may refer to the ability of a liquid or solid medium retaining structure to hold or store that medium).

The retaining material is designed to react to the force applied by expansion of the balloon 108. When the balloon is in deflated state, the pores are closed under the compression that naturally exists within the property of the material, effectively retaining the agent/drug therein. However the force with which the expanded condition of the balloon exerts radially, will un-compress the pores, releasing therapeutic agents to the site. In many instances, varying such material characteristics, including but not limited to: tensile strength, stiffness, Young's Modulus, etc., may vary the force applied by the balloon expansion. One skilled in the art can design a retaining material with particular desired characteristics to un-compress by the force that is applied when balloon reaches a specific diameter. For example, when treating a 3 mm vessel diameter, the porous surface un-compresses only when the balloon expands to that specific diameter, thereby preventing premature infusion, diffusion and maintaining the original drug load during delivery and inflation of the device.

Further examples of devices and methods which may be utilized and integrated with the systems described herein are shown and described in farther detail in. U.S. patent application Ser. No. 11/461,764 filed Aug. 1, 2006, which is incorporated herein by reference in its entirety.

Once the catheter system 100 has been advanced and desirably positioned within the vessel to be treated, the agents/drugs contained within the outer retaining surface 112 may be applied to or against the interior of the vessel to be treated, as further described below.

Although a single balloon 108 is illustrated, one or more balloons positioned in series relative to one another may alternatively be utilized. Each of the balloons may be connected via a common inflation and/or deflation lumen to expand each of the expandable members. Alternatively, each of the balloons may be connected via its own inflation/deflation lumen such that individual balloons may be optionally inflated or deflated to treat various regions of the vessel.

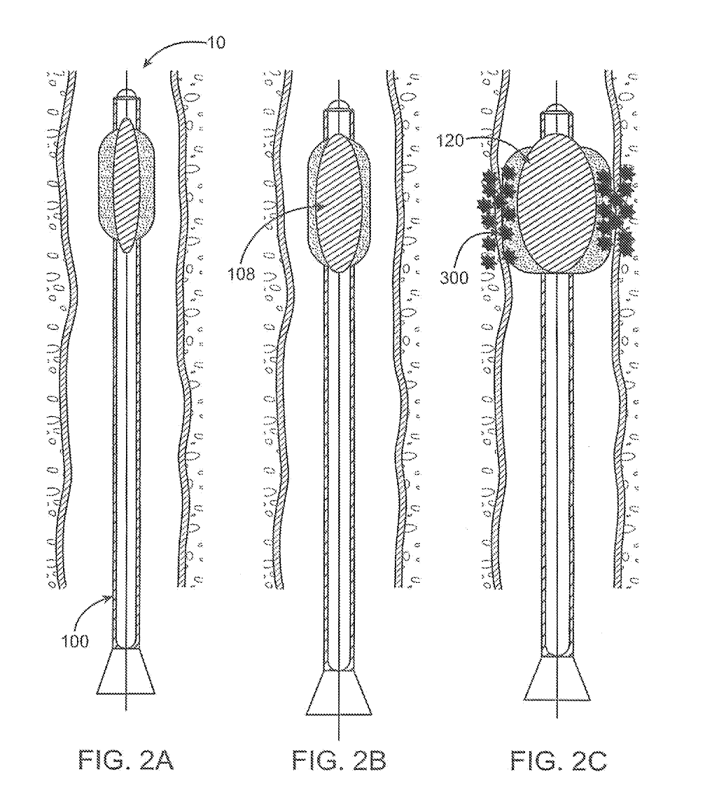

FIG. 2A shows the catheter assembly 100 introduced into the vessel and advanced to the location to be treated. Once desirably positioned adjacent to or proximate to the vessel 10 to be treated, the balloon 108 may be inflated via pump 14 through inflation/deflation tube 12, as shown, in FIG. 2B, and appose its porous surface 112 uniformly or otherwise against the interior wall of the vessel 10. Pressure from the balloon 108 will un-compress pores of the surface 120, causing release of the agents 300, directly, uniformly (or non-uniformly), and efficiently to the vessel with minimum dilution and diffusion, shown in FIG. 2C.

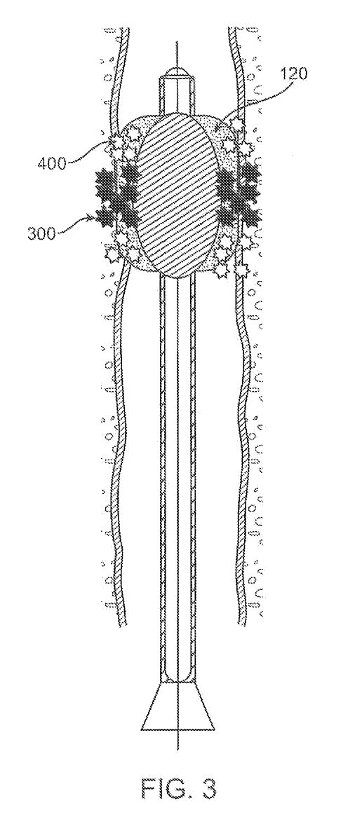

Once the desired agents/drugs have been applied for a desired period of time, the catheter system 100 may be deflated and removed from the vessel. FIG. 3 show another variation of the retaining surface 112, having one drug agent 300 at its center and different agents or different concentration of the same agent/drug 400 at its proximal and distal ends to address “edge effect re-stenosis”.

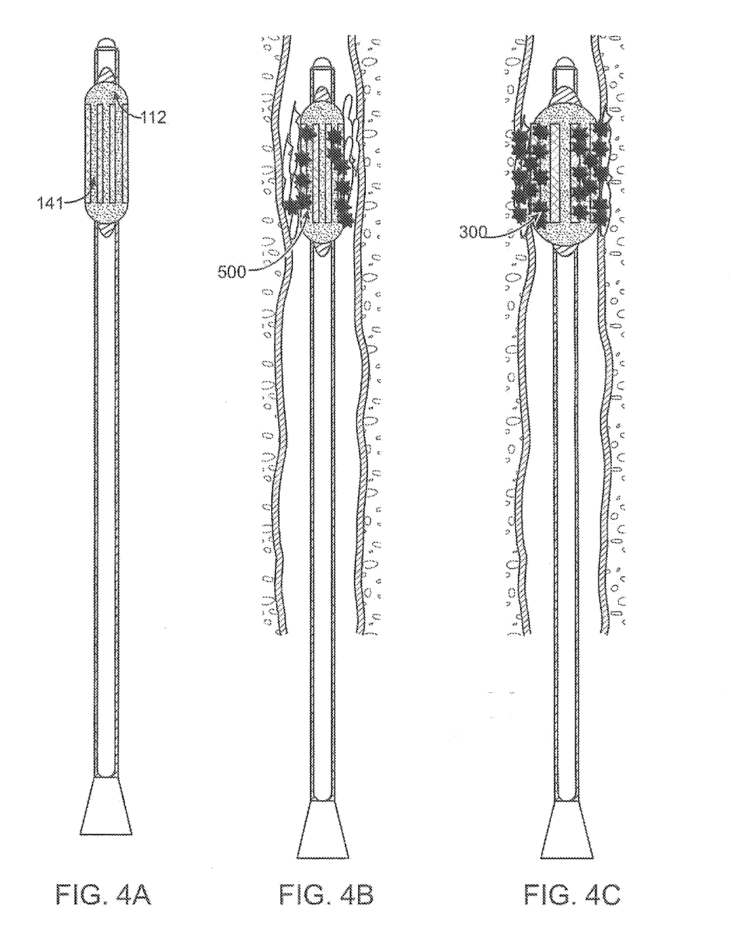

FIG. 4A illustrates yet another variation of the retaining surface 112, capable of releasing different agents or different concentration of the same agent at different balloon diameters. This is accomplished by the porous surface 112 having longitudinal sections 141 capable of un-compressing when balloon is inflated to its first diameter, thereby releasing the first drag that in present example is a fibrolytic agent 500 to dissolve the thrombus within the stenotic lesion of tire vessel as shown in FIG. 4B. Further inflation of the balloon will un-compress the remaining segments of the porous surface that contain anti-restenotic agent 300 and release such agents to the vessel wall, shown in FIG. 4C.

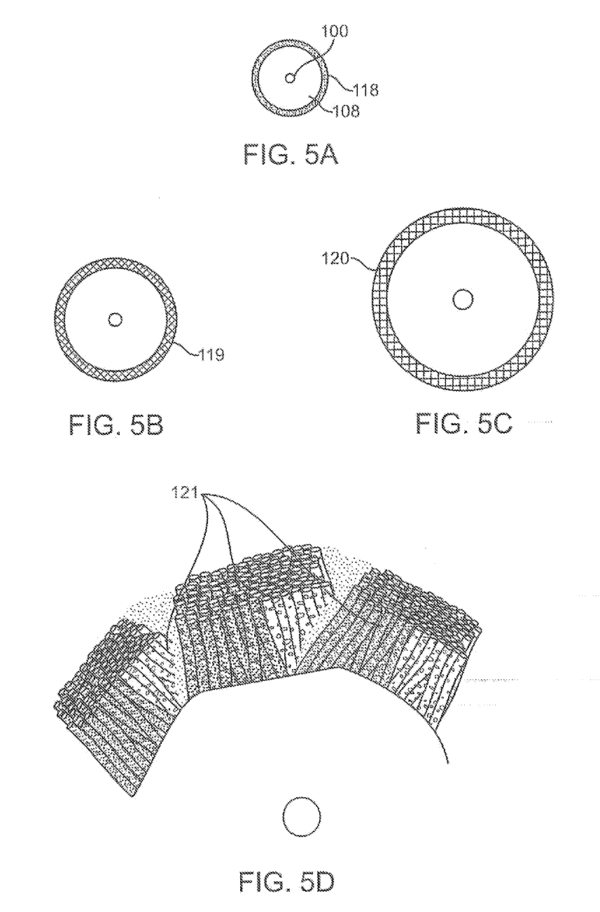

As shown in FIG. 5A, the treatment device 100 is a balloon 108 coupled with an outer porous layer 118. The treatment device 100 is positioned such that the balloon 108 coupled with outer porous layer 118 is adjacent to the target lesion. The assembly may then be inflated and expanded as shown in FIGS. 5B and 5C by infusing an inflatable agent such as saline. As the assembly is inflated and expanded, the outer porous layer 119 is stretched. As shown at FIG. 5C, the initial pore configuration may then be changed while the balloon remains inflated, causing the biological substance entrapped in the cell of the stretched or otherwise deformed porous layer 120 to become available for the contact with a targeted tissue.

Further variations may include a microporous cross-linked polymer matrix having a predetermined pore architecture. A “pore” may include a localized volume of the outer layer that is free of the material from which the outer layer is formed. Pores may define a closed and bounded volume free of the material from which outer layer is formed. Alternatively, pores may not be bounded and many pores may communicate with one another throughout the internal matrix of the present outer layer. The pore architecture, therefore, may include closed and bounded voids as well as unbounded and interconnecting pores and channels. The internal structure of the outer layer defines pores whose dimensions, shape, orientation and density (and ranges and distributions thereof), among other possible characteristics are tailored so as to maximize the capacity of the treatment device to contain and deliver under pressure certain, biological substances. There are numerous methods and technologies available for the formation matrices of different pore architectures and porosities. By tailoring the dimensions, shape, orientation and density of the pores of the outer layer, a capacity to absorb and release biological agents in certain predictable manner may be formed that may be used for local drug delivery.

An embodiment of the outer layer may be formed of or include a polyurethane matrix having a predetermined pore architecture. For example, the outer layer of the treatment device may include one or more sponges of porous polyurethane having a predetermined pore architecture. Suitable polyurethane material for the outer layer of the treatment device maybe available from, for example, Lendell Manufacturing, Inc.: Hi-Tech Products (Buena Park, Calif.), PAC Foam Products Corp. (Costa Mesa, Calif.), among others. Moreover, the outer layer may be comprised of any number of suitable materials including, but not limited to, elastomeric and non-elastomeric polymers such as polyurethane, silicone, pebax, polyimide, polyethylene, polyetheretherketone (PEEK), polyvinylidene fluoride (PVDF) liquid crystal, polymer (LCP), family of fluoropolymers such as polytetrafluoroethylene (PTFE), expanded polytetrafluoroethylene (ePTFE), family of polyesters such as Hytrel, Polyethylene terephthalate (PET), polybutylene terephthalate (PBT) and their copolymers, etc. The outer layer of the treatment device may, according to further embodiments, be used to medically treat the patient. That is, the porous matrix of the outer layer may be imbibed or loaded with a therapeutic agent to deliver the agent through elution at the interior of the vessel wall. Such a therapeutic agent may include, for example, biopharmaceuticals, therapeutic agents or physiological process modifying agents which can be anti-infective, anti-inflammatory, anti-proliferative, anti-angiogenic, anti-neoplastic, anti-scarring, scar-inducing, tissue-regenerative, anesthetic, analgesic, immuno-modulating agents and neuro-modulating, bioadhesives, tissue sealants and sclerosing agents, to name but a few of the possibilities.

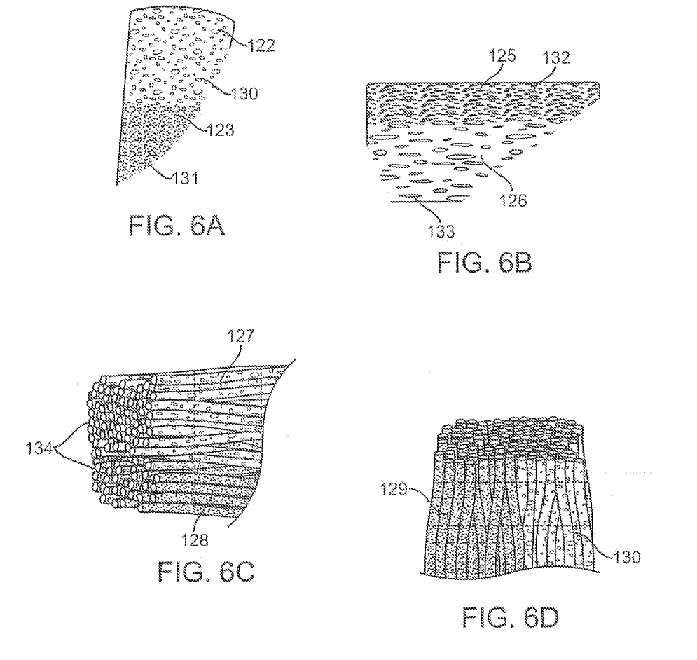

The outer layer 121 shown in FIG. 5D may be formed of one or more thin sheets or fibers of polyurethane or silicone material having a predetermined (and controlled) pore architecture that has been coupled with the outer surface of the balloon. FIG. 6A shows an outer layer 121 having predetermined pore architectures. As shown therein, the outer layer 121 may include a first portion 122 and a second portion 123. The polymer matrix of the first portion 122 of the outer layer 121 defines a plurality of pores 130 having a first predetermined pore architecture and the polymer matrix of the second portion 123 of the outer layer 121 defines a plurality of pores 131 having a second predetermined pore architecture. The dimensions of the layers or portions may be selected at will, preferably accounting for the dimensions of the treatment device. As shown, the first pore architecture features pores 131 that are relatively small, have a narrow pore size distribution and are substantially randomly oriented. In contrast, the second pore architecture features pores 130 that have a relatively larger size, have a wider pore size distribution, and are less densely distributed than the pores 130 of the first portion 122 of the outer layer of the treatment device.

FIG. 6B shows a segment of outer layer 121 having an alternative predetermined pore architecture. As shown, the outer layer includes a first portion 125 and a second portion 126, each of which has a predetermined pore architecture (pore 132 in first portion 125 and pore 133 in second portion 126, which in this example illustrates pores 132 having a smaller size relative to pores 133). It is to be noted that the present outer layer may have more than the two portions. The first portion 125 is stacked on the second portion 126. As with the embodiment shown in FIG. 6A, the first and second portions may have pore architectures that facilitate optimal drug absorption characteristics. The different pore architectures of the outer layer may also be chosen so as to maximize the controlled drug release when the balloon 108 is fully expended and positioned against the targeted lesion.

FIGS. 6C and 6D show various other configurations for the porous outer layer. As shown therein, embodiments may include or be formed of a bundle of fibers or fibrils 134 of (for example) polyurethane material having one or more predetermined pore architectures. The pores 127, 138, 129 defined within the polyurethane matrix of all or some of the fibers are shown in the various figures herein.

As shown in FIG. 6C, two or more bundles of fibers of polyurethane material (for example, the fibers maybe made of or include other materials) may be used in the formation of outer layer. As shown, the pores within the fibers of the first bundle 127 may collectively define a first pore architecture, whereas the pores within the fibers of a second bundle 128 may collectively define a second pore architecture that is different from the first pore architecture. The two bundles may then be joined together, for example, by re-wetting the bundles, stacking them and lyophilizing the composite structure. The length and diameter of the fibers maybe selected and varied at will. The fibers or bundles thereof may even be woven together. From this composite structure, outer layer may be formed. As shown in FIGS. 6C and 6D, the bundles of fibers maybe arranged and oriented in a different manner for example perpendicular or parallel to the surface of the balloon.

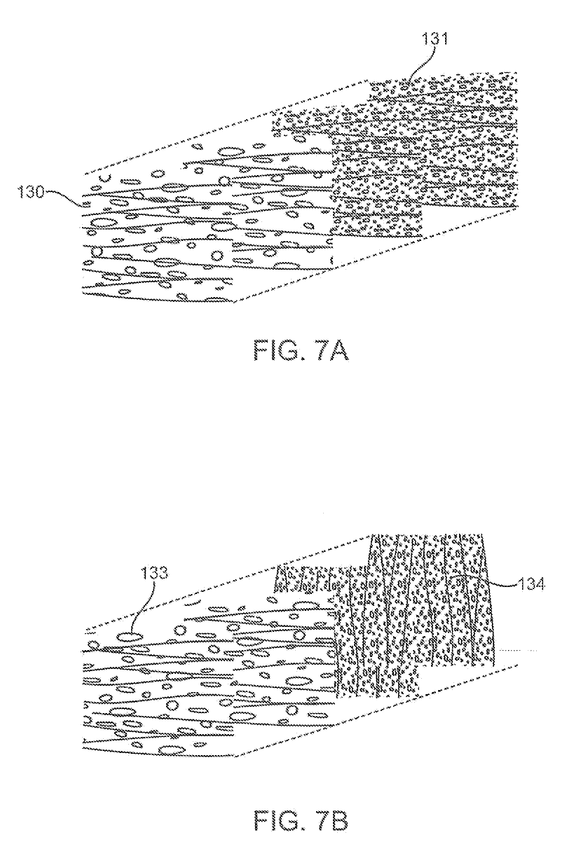

As shown in the exploded views of FIGS. 7A and 7B, the outer layer may have a layered laminate structure in which sheets formed of fibers (or woven fibers) having a first porearchitecture are stacked onto sheets formed of fibers having a second pore architecture. As shown in FIG. 7A, many variations on this theme are possible. As shown therein, the orientation of the fibers (and thus of the pores defined by the polymer matrix thereof) may be varied. For instance, whereas the fibers of the first (top or outer, for example) portion of the outer layer may be oriented in a first direction, whereas the fibers of the second (bottom or inner, for example) portion of the outer layer may be oriented along a direction that is different from the first direction (perpendicular thereto, for example).

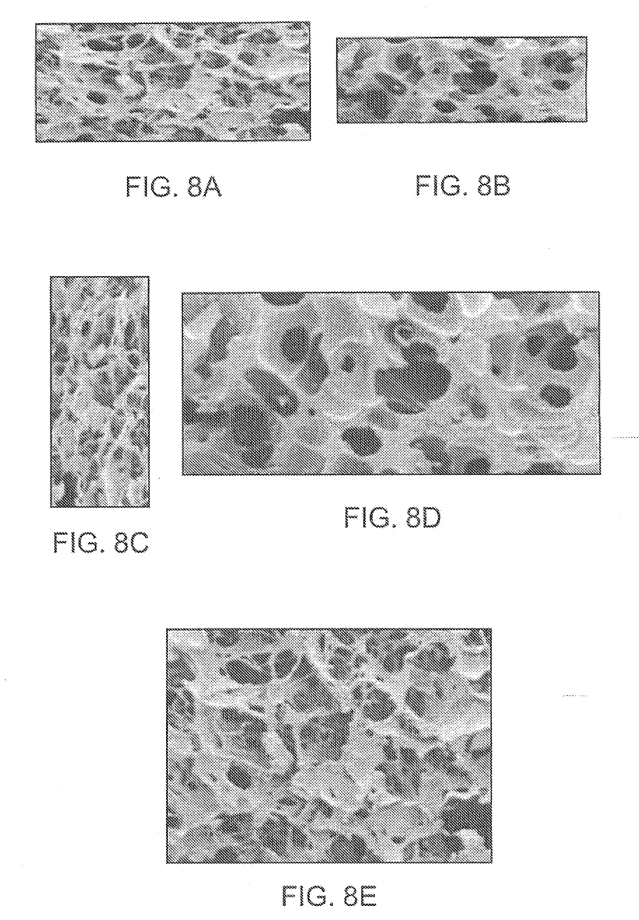

FIGS. 8A to 8E are photomicrographs of polymeric matrices having various pore architectures that can be generated using various technologies such as lyophilization or usage of a foaming agents, just to mention a few.

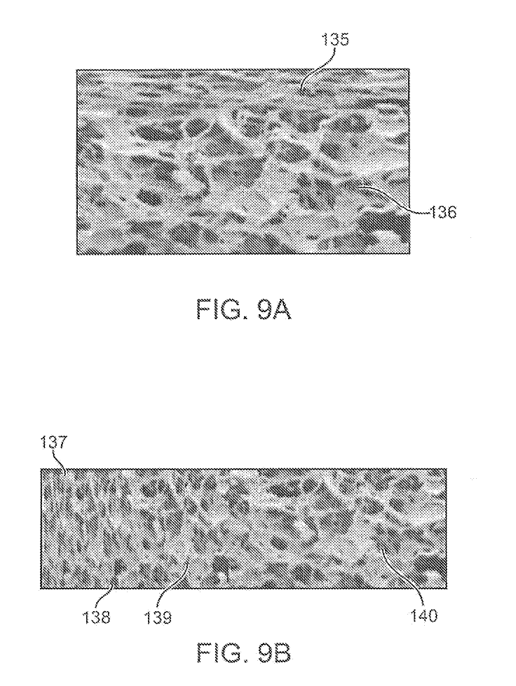

FIGS. 9A and 9B are combinations of photomicrographs to illustrate further embodiments of the outer layer. FIG. 9A shows an outer layer 121 that includes a first portion 135 having a first pore architecture and, stacked thereon, a second portion 136 having a second pore architecture. As shown, the pore architecture of the first portion 135 may be characterized as being relatively denser than the pore architecture of the second, portion 136. Alternatively, the outer layer 121 may be structured such, that the first portion has a higher porosity (is less dense) than that of the second portion 136. The thicknesses of the first and second portions 135, 136 may be varied at desired. More than two layers of polymeric material may also be provided.

FIG. 9B shows an outer layer 121 having a graduated porosity profile. Such an outer layer may be formed by lining up a plurality of polymer matrices having of progressively lower densities. That is, matrix 137 has the highest density (amount of polymer per unit volume), matrix 138 has the next highest density, matrix 139 has the next to lowest porosity and matrix 140 has the lowest porosity of the entire outer layer.

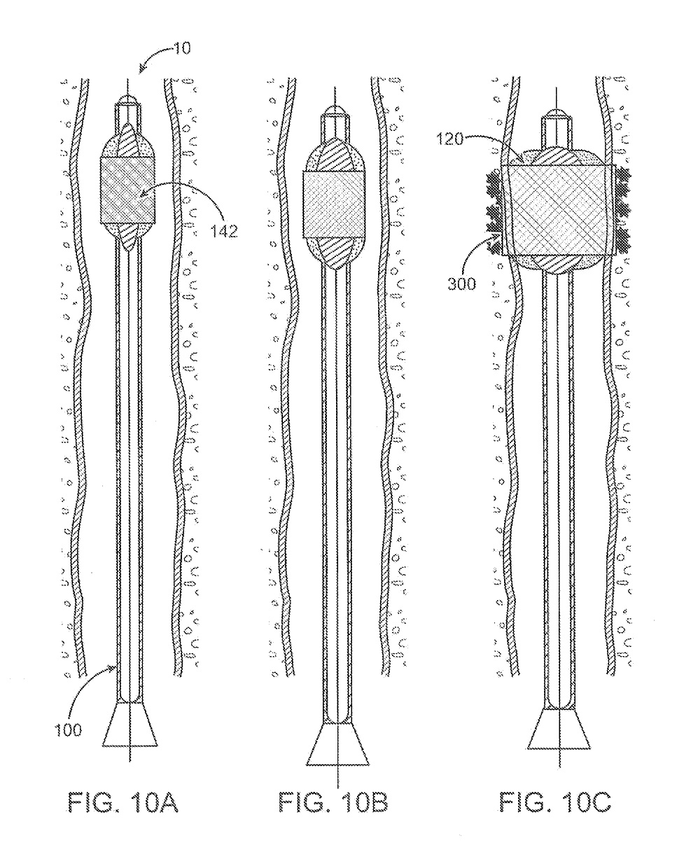

FIGS. 10A to 10C show the treatment device 100, delivering a stent 142 and simultaneously infusing a therapeutic agent into the targeted site.

A three-dimensional internal geometry and capability for retention or release of its contents is desirable. Such retention or release of substances are dependent on the type of application and the amount of the hoop stress required for the substrates in order to provide an effective local drug delivery of a prescribed dose to a targeted tissue. The substrate can be built or coupled to the surface of the balloon or produced in the form of a sleeve that can be fitted upon the balloon. Such porous substrate sleeves can be processed by several techniques well known in the fields of polymer processing and tissue engineering.

One of the methodologies of formation of porous polymer structures involves the mixing of water soluble inorganic salts into polymer-solvent systems and forming a tubular structure of a desired but limited thickness by one of many procedures available. The resulting polymer network is then cured and leached of salt by soaking in an aqueous solution.

Yet another method for forming a porous polymer substrate sleeve involves freezing water dispersion of a polymer at a certain regime so that water crystals of a certain size and shape are formed. The resulting frozen polymer network is then freeze-dried and water crystals are sublimated by application of a vacuum.

Also, foaming agents such as cyclopentane and blowing agents such as certain chlorofluorocarbons (CFCs), just to mention a few, can be used to produce “pseudo-porous structures”, i.e., to produce a closed pore cellular structure to the polymeric substrate sleeve.

Yet another method for forming a porous polymer substrate sleeve is utilization of mandrel dipping. Mandrel dipping methods can result in substrates which are limited to simple, thin-walled porous substrate material. Reproducibility and uniformity of the porous structures formed by dipping is typically tightly controlled.

Yet another method for forming a porous polymer substrate can utilize certain techniques similar to those employed for a formation of a porous graft particularly adapted for cardiovascular use, as described in U.S. Pat. No. 4,759,757 entitled “Cardiovascular graft and method of forming same”, which is incorporated herein by reference in its entirety. The described method generally comprises choosing a suitable, non-solvent, two component, hydrophobic biocompatible polymer system from which the graft may be formed; choosing suitable water soluble inorganic salt crystals to be compounded with the biocompatible polymer system; grinding the salt crystals and passing same through a sieve having a predetermined mesh size; drying the salt crystals; compounding the salt crystals with the biocompatible polymer system; forming a tube from said compounded salt and polymer system by reaction injection or cast molding; and leaching the salt crystals from the formed tube with water, said leaching of said salt crystals providing a tube with a network of interconnecting cells formed in the area from which the salt crystals have been leached.

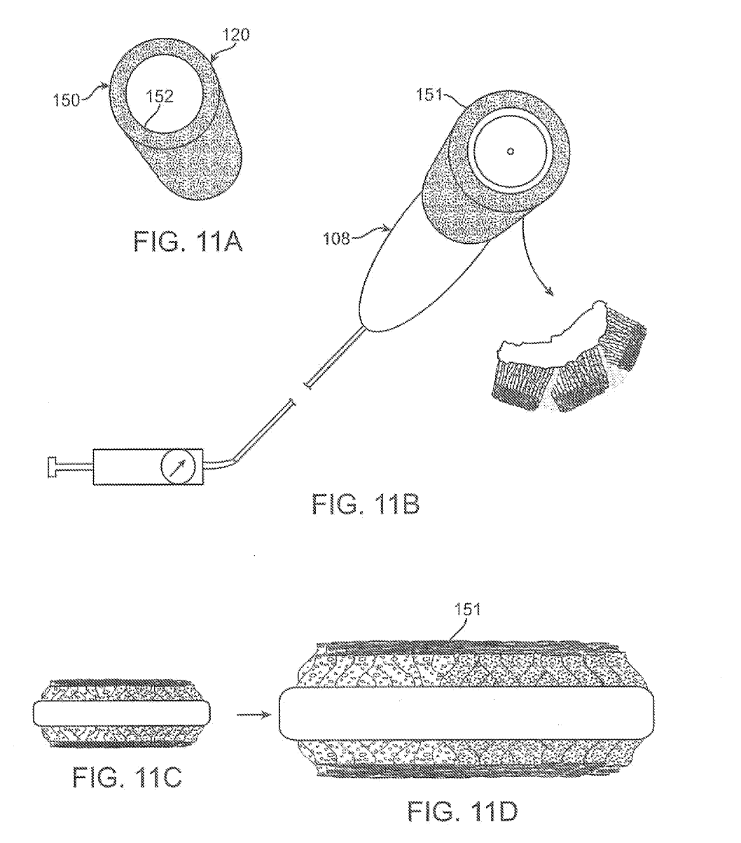

All of the above methods are suitable for the three-dimensional substrates manufacturing. Now referring to the drawings in greater detail, a sleeve 150 is illustrated in FIG. 11A which has a tubular configuration within an inner surface 152 and an outer surface 151 and is formed of a porous biocompatible polymer material with the surface 152 and 151 having cells or pores 120 therein. Referring now to FIG. 11B, there is illustrated a perspective view of the substrate sleeve 150 introduced upon the balloon 108 and a side view in FIG. 11C.

Referring now to FIG. 11D, there is illustrated therein an enlarged longitudinal view of the substrate sleeve fitted on the balloon. In this view is illustrated the honeycomb arrangement of the cells or pores 120. In this respect, by forming the sleeve 150, the cells or pores 120 within the sleeve are formed so that they interconnect throughout the wall thickness to form a porous network through the wall to the sleeve 150. This honeycomb network arrangement in a porous biocompatible polymer facilitates elution of a loaded biological substance into a substrate upon applying a certain hoop stress by the inflated balloon 108.

Referring now one of the suggested method for forming the substrate sleeve 150, it is first to be noted that the biocompatible polymer system from which the substrate sleeve is manufactured is a two component polymer system including polymers such as polyurethane, silicone and polytetrafluorethylene and a curing agent. Also, other hydrophobic polymer systems may be utilized and the choice of materials should not be confined to these three polymers. In such a two component polymer system, the first component is a resin, such as a silicone resin, and the second component is a curing agent/catalyst such as, for example, platinum. Other curing agents/catalysts available for use in such two component systems are tempered steel, heat, crosslinkers, gamma radiation, and ureaformaldehyde. As described above, it will be noted that this two component system is a non-solvent system. That is, the two components react together in the presence of salt, which is compounded with the two component system as described below. The two components are not a polymer and a solvent.

Once an appropriate two component polymer system has been chosen, it is compounded with a water soluble inorganic salt such as, but not confined to, sodium chloride. The size and shape of the pores 120 of the honeycomb network are dictated by the choice of the specific inorganic salt that is compounded with the polymer system. Typically, the crystals of salt chosen are ground and then put through a sieve whose chosen mesh size corresponds to the size requirement for the pore diameter to be utilized in the graft 10. The salt crystals are then placed in a drying oven at 135° C. for a period of, e.g., no less than 24 hours. The polymer system is then processed according to the method recommended by the manufacturer of the particular polymer system utilized and the dried salt crystals are mixed with the polymer system and compounded. The porosity and flexibility of the substrate sleeve 150 is dependent upon the ratio of water soluble inorganic salt to the polymer system with this ratio ranging anywhere from 25-755 by weight.

Once compounded, the water soluble inorganic salt and polymer are injection molded or reaction injection molded to form a tube of known inner and outer diameter. If desired, the tube can be extruded. Once the salt filled polymer tubes are formed, they are leached in water, dissolving the salt crystals and leaving a porous network of interconnecting cells 151, as Illustrated in FIG. 11D. This method of formation provides for the rapid and reproducible formation of simple geometries within thin walled substrate sleeves as well as large, intricate geometries within thick walled substrate sleeves as dictated by the size of the anatomical structures in which the substrate sleeves is to be utilized.

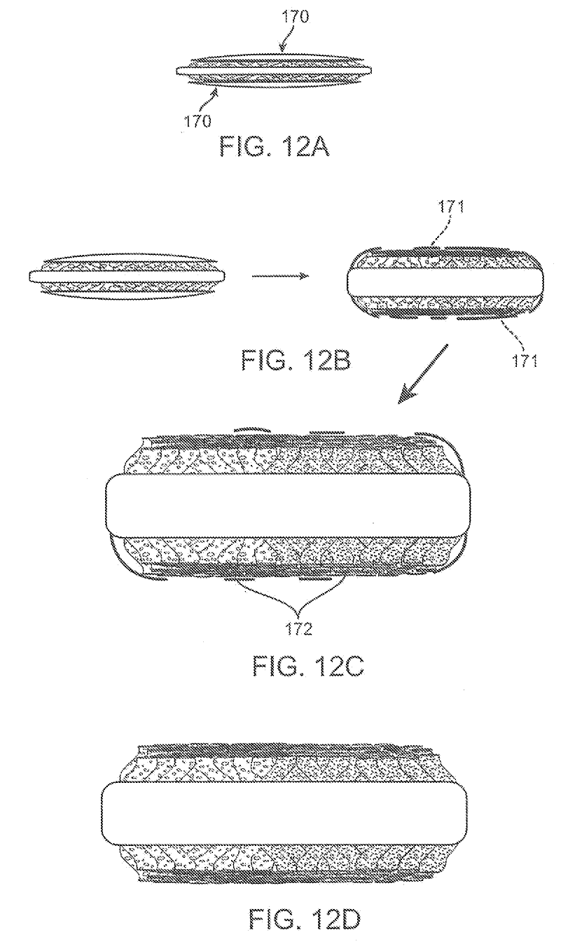

FIGS. 12A to 12D illustrate yet another embodiment, where a thin layer of a polymeric biodegradable film 170 is placed on the outer surface of the substrate sleeve thereby preventing any undesirable leakage of the biologically active substance coupled with the substrate. FIG. 12B illustrates the expansion of the balloon and as a consequence of that disintegration and defragmentation of the coating film 170 turning it into a disintegrated surface 171. Biodegradable coating 170 can be formed with a variety of the biopolymers such as, but not limited to, synthetic and naturally occurring polymers including hydrophilic and hydrophobic synthetic polymers, small molecular weight crosslinkers having at least two carbon atoms, proteins, polysaccharides, lipids, DNA and their derivatives. Hydrophilic polymers may include, but are not limited to: polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propyene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimetylene glycol; polyoxyethylated sorbitol, polyoxyethylated glucose; acrylic acid polymers and analogs and copolymers thereof, such as polyacrylic acid per se, polymethacrylic acid, poly(hydroxyethylmethacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such, as poly(vinly alcohol); poly(N-vinyl lactams) such as polyvinyl pyrrolidone), poly(N-vinyl caprolactam), and copolymers thereof; polyoxazonines, including poly(methyloxazoline) and poly(ethyloxazoline); and polyvinylamines. Naturally occurring hydrophilic polymers may include, but are not limited to: proteins such as collagen, fibronectin, albumins, globulins, fibrinogen, and fibrin, with collagen particularly preferred; carboxylated polysaccharides such as polymannuronic acid and polygalacturonic acid; animated polysaccharides, particularly the glycosaminoglycans; e.g., hyaluronic acid; chitin chondroitin sulfate A, B, or C, keratin sulfate, keratosulfate and heparin; and activated polysaccharides such as dextran and starch derivatives, etc.

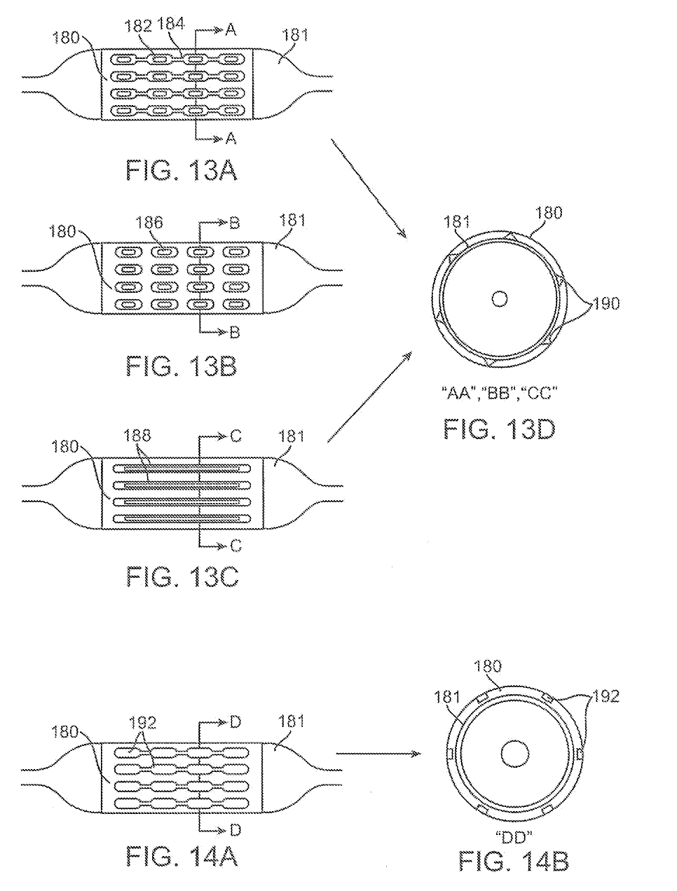

FIGS. 13A to 13C illustrate, additional variations of the expandable sleeve 180 placed upon an expandable balloon 181 and which have various configurations for reservoirs along the sleeve surface which are capable of expanding when the balloon 181 reaches a predetermined diameter to release any biologically active substances. One example is illustrated in the perspective view of FIG. 13A where a plurality of individual reservoirs 182 interconnected via channels 184 may form a network of reservoirs over the sleeve surface. The individual reservoirs 182 may be uniformly spaced over the sleeve surface or scattered in various patterns depending upon the desired release results. Another variation is shown in FIG. 13B which illustrates a plurality of independent reservoirs 186 spaced over the sleeve surface uncoupled from one another. Yet another variation is illustrated in FIG. 13C which illustrates a variation where reservoirs 188 are configured to extend longitudinally along the surface of sleeve 180. Although the reservoirs are illustrated as being formed upon the sleeve 180 which is placed upon balloon 181, the reservoirs may be alternatively formed directly upon the balloon surface rather than upon a separate sleeve 180.

In forming the reservoirs, several manufacturing methods such as micro machining, chemical etching, ablation (laser, ultrasound, RF, microwave, electron beam), selective laser sintering, etc., as well as various other polymer processing methods such as dip coating, injection molding, etc., can be utilized to create these reservoirs. Moreover, the geometries of the reservoirs may be designed in such a manner to provide for significant dose capacity, prevent premature release, and enable sufficient expansion in radial direction, thus effective drug release is achieved upon expansion of the balloon. This may be achieved, e.g., by forming the reservoirs 190 in a conical or angled configuration in the outer layer where each reservoir 190 may have a wider base adjacent to the balloon 181 surface and angle to a closed configuration as reservoir 190 extends radially away from balloon 181, as illustrated in the representative cross-sectional view of FIG. 13D. With balloon 181 in a deflated configuration, the apex of reservoirs 190 maybe closed upon itself to contain the biological agent. However, as balloon 181 is expanded, the apex of reservoirs 190 may open to release the agents contained within.

Another variation is illustrated in the perspective view of FIG. 14A, which shows interconnected reservoirs 192 defined along the surface of balloon 181. The cross-sectional profile of FIG. 14B shows each reservoir 192 configured as a pore or well shape to which the agent may be added as a viscous fluid to facilitate its insertion and packing into the pores or reservoirs 192 of the outer surface. The thermal property of the viscous fluid is selected in a manner to cause significant reduction in the viscosity upon its exposure to the body temperature. This will further enhance drug transport into the tissue, when the balloon reaches its maximum diameter and brings the drug containing fluid in contact with the blood vessel.

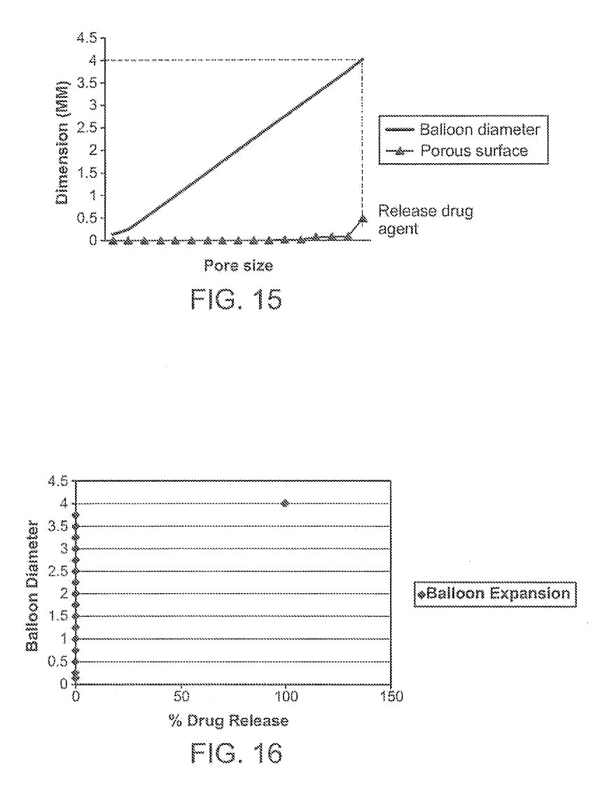

FIG. 15 is a graph showing an increase in pore size and correlated release of a drug agent when the balloon reaches its maximum diameter. This illustration is an example of a balloon diameter of, e.g., 4 mm, coupled with a porous surface with stretched pore size of, e.g., 0.5 mm.

FIG. 16 is a graph showing the complete release of a drug agent at a predetermined balloon diameter of, e.g., 4 mm.

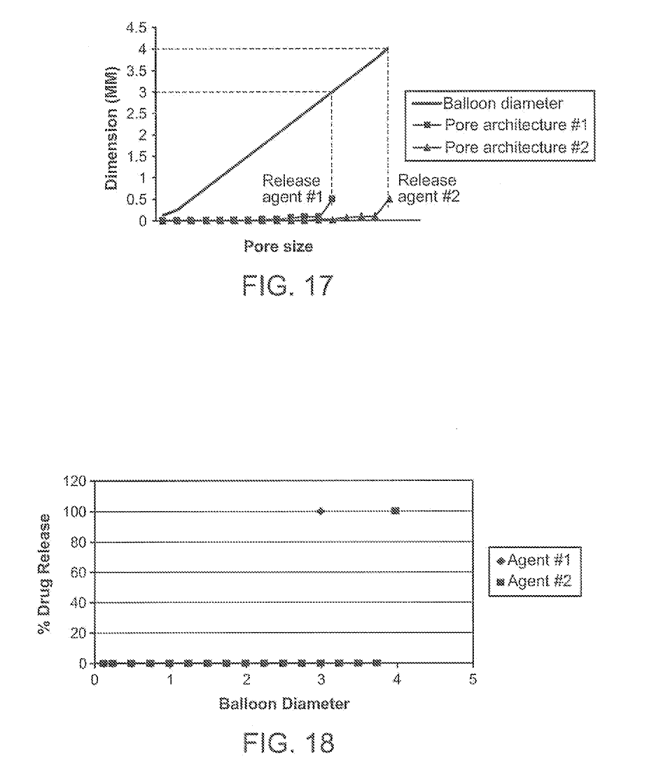

FIG. 17 is a graph showing an example of two different pore architectures responding to the balloon expansion. When the balloon reaches its first diameter of, e.g., 3 mm, pores of the first architecture open, causing the release of the first drug agent. Full inflation of the balloon to 4 mm diameter will open the pores of the second architecture, causing the release of the second drug agent.

FIG. 18 is a graph showing 100% release of a first drug agent when balloon researches its first diameter of, e.g., anywhere from 1 mm to 5 mm and particularly to 3 mm, followed by complete release of a second drug agent when the balloon is fully inflated to, e.g., anywhere from 5 mm to 10 mm and particularly to 4 mm in diameter.

Although various diameters for an inflatable balloon are described, these examples are illustrative of balloon inflation and an inflatable balloon as utilized herein may be inflated to any suitable diameter, e.g., 1 mm to 10 mm, for effecting a treatment.

In intravascularly advancing a balloon catheter having the porous outer layer disposed thereupon, an outer sheath may be used to cover the porous layer during delivery through the vasculature to retain any biologically active substances or agents placed, infused, or otherwise disposed within or upon the outer layer. However, the cross-sectional size of the sheath may undesirably increase the diameter of the balloon and porous outer layer, particularly for neurovascular applications where the vessels are tortuous and relatively small in diameter. Moreover, retraction of a sheath from the porous outer layer may be difficult depending upon the tortuous configuration of the delivery catheter. Furthermore, retracting the sheath may also undesirably remove some of the agent placed, infused, or disposed upon the porous outer layer. Delivery of the porous outer layer assembly without a sheath may also release undesirable amounts of the agent disposed within or upon the outer layer into the vasculature and any therapeutic amounts of agent upon the outer layer may also be diluted by the time the targeted tissue region is reached.

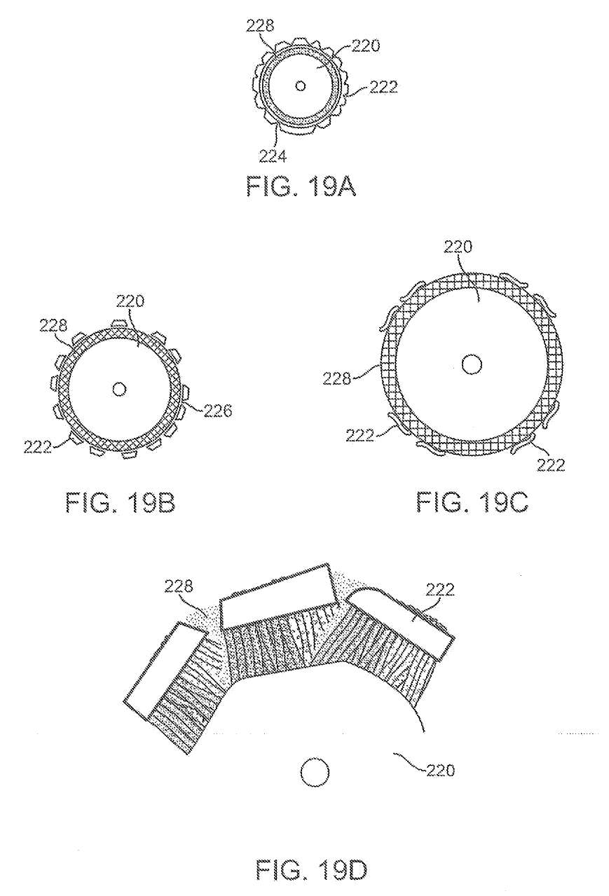

Accordingly, in one variation as shown in FIG. 19A, outer porous layer 228 disposed upon balloon 220 may be coated or otherwise encapsulated by a structurally jeopardized or weakened outer sheath 222. Outer sheath 222 may retain any biological agents placed or infused upon or within the outer layer 228 while maintaining a low-profile diameter of the assembly. The outer sheath 222 may be weakened by any number of mechanical discontinuities, e.g., using various techniques such as creating scores, notches, and/or cuts 224 along its surface so that when the balloon 220 is coupled with outer porous layer 228 and inflated, outer sheath 222 may be easily split or fragmented. 226 along the weakened portions 224 of outer sheath 222 in a predictable manner due to the imparted radial stresses, as shown in FIG. 19B. Examples of materials which may be utilized for fabricating the outer sheath 222 may include, but not limited to, e.g., polysaccharides, hyaluronic acid (HA), alginates, PEG, PEA, PGA, PGA-PLA copolymers, or any of the other suitable materials described herein.

The balloon 220 and outer porous layer 228 may be further inflated and expanded, as shown in FIG. 19C, such that the outer sheath 222 is further stretched and ultimately disintegrated or decoupled from the porous layer 228. While the balloon 220 remains inflated, the biological agents entrapped in the cells of the stretched or otherwise deformed porous layer 228 may be exposed for contact with and delivery to a targeted tissue, as described above. FIG. 19D shows an enlarged cross-sectional view of the inflated balloon 220 coupled with porous layer 228 partially covered with remaining portions of disintegrated outer sheath 222. Utilizing a sheath 222 which disintegrates upon expansion of the balloon 220 eliminates complications relating to sheath retraction and also maintains a low-profile of the outer layer 228 as a thin layer of the outer sheath 222 may be used. Although the thickness of outer sheath 222 may be varied to suit different applications, the thickness may generally range anywhere from 1 μm to 500 μm.

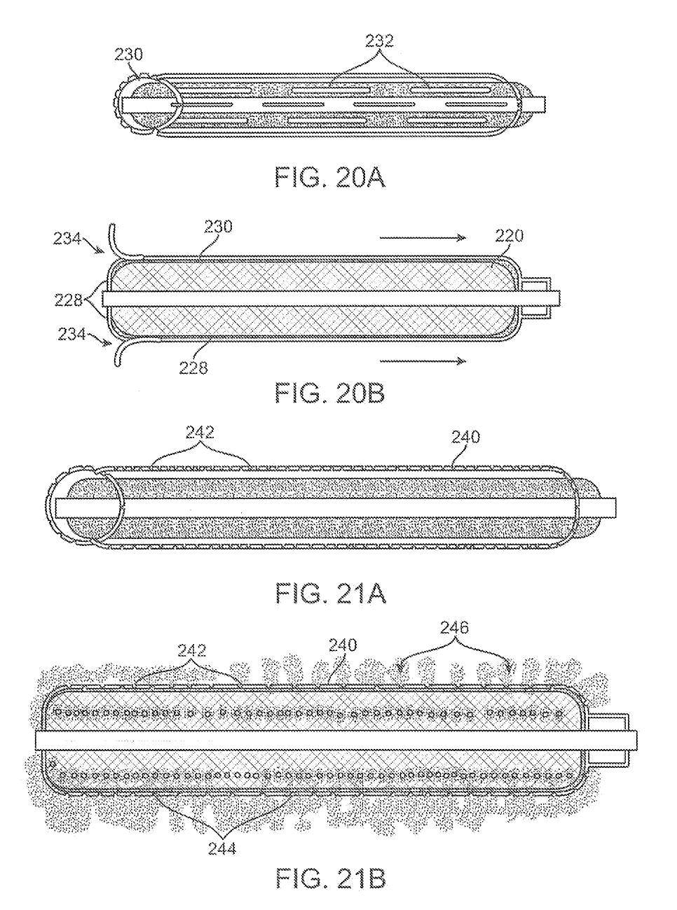

FIG. 20A shows another variation where the balloon may be covered with a structurally jeopardized outer sheath 230 where the sheath surface is weakened by multiple longitudinal grooves or cuts 232. Upon expansion of the balloon 220, outer sheath 230 may be unsheathed or ruptured due to the radial stresses imparted by the balloon 220. In the example of FIG. 20B, outer sheath 230 is illustrated rupturing initially at its distal end 234 to expose the underlying porous outer layer 228.

FIG. 21A shows a side view of yet another variation of a disintegrating outer sheath 240 which is structurally jeopardized by a plurality of perforations or holes 242 formed throughout the surface of outer sheath 240. The hole diameters may range individually or uniformly anywhere from 1 μm to 300 μm. As the balloon is inflated the perforations or holes 242 may become significantly increased in diameter 244 allowing the biological agent 246 to be released or available for treatment upon the targeted tissue, as shown in FIG. 21B. Alternatively, outer sheath 240 may begin to disintegrate along the perforations or holes 242 as the balloon is inflated to expose the underlying porous outer layer 228 for treatment.

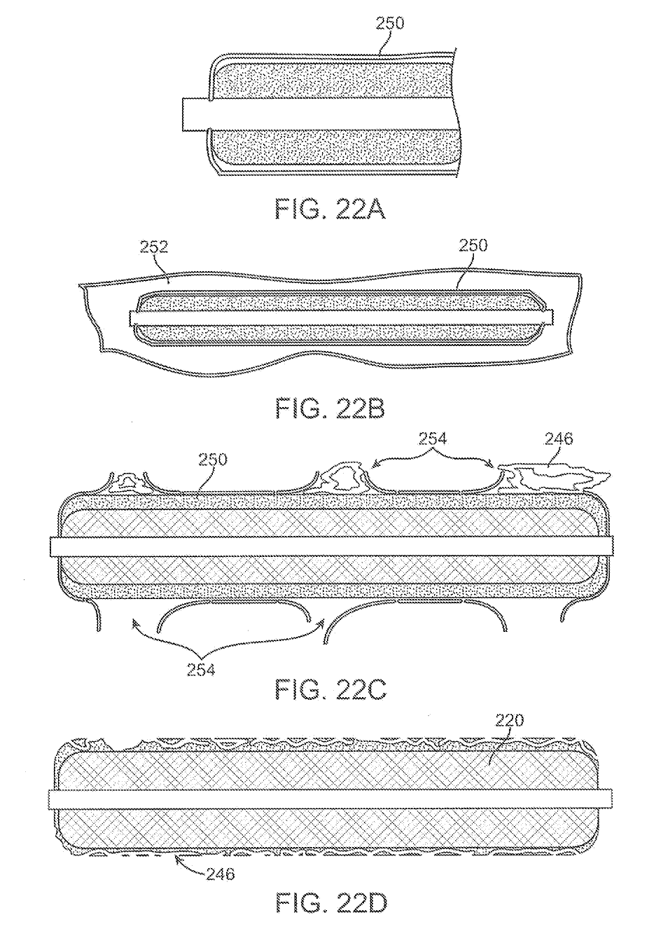

FIGS. 22A to 22D illustrate yet another variation where a thin layer of a structurally jeopardized polymeric biodegradable film 250 is placed on the outer surface of the porous outer layer to prevent any undesirable leakage of the biologically active substance coupled with the substrate. Biodegradable coating 250 can be formed a variety of the biopolymers such as, but not limited to, polysaccharides, hyaluronic acid (HA), alginates, PEG, PLA, PGA, PGA-PLA co-polymers, starch, sucrose, fructose, chitosan, or any other suitable materials described herein, etc. As shown in FIG. 22B, when placed in the blood stream 252 the thin layer of biodegradable film 250 be dissolve and become completely disrupted upon full inflation of the balloon to create gaps or openings 254 along the film 250 and thus releasing biological agents 246 contained in the underlying porous outer layer, as shown in FIG. 22C. Disintegrated fragments of such a biocompatible and biodegradable film 250 will be easily dissolved in the blood stream and metabolized. Once the film 250 has been disintegrated or otherwise dissolved, the inflated balloon 220 and outer porous layer may remain to release the biological agents 246, as shown in FIG. 22D.

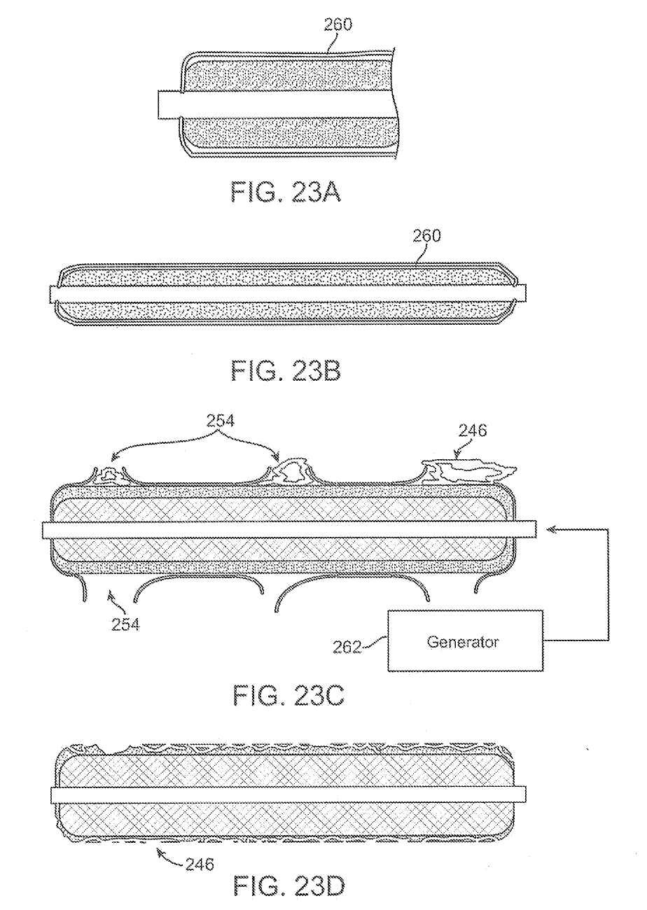

In yet another variation, the outer sheath may comprise a metallic erodable membrane 260 that may seal and/or encapsulate the porous outer layer and balloon assembly, as shown in FIGS. 23A and 23B. The metallic membrane 260 may be in electrical communication through the delivery catheter with a power supply, e.g., DC power generator 262, located externally of the patient body, as shown in FIG. 23C. Examples of suitable metallic materials which may be utilized as a membrane 260 may include, but are not limited to, e.g., Stainless steel, Magnesium alloys, NiTi alloys (Nickel-Titanium), Platinum, Platinum alloys, Gold, etc. The membrane 260 may be attached to a positive terminal while the patient is connected to a negative terminal of the DC power generator 262 such that, when the balloon is expanded, a small amount of current may be applied to positively charge the metallic membrane 260 and negatively charge the patient. This electrical potential difference creates electrolysis between the membrane 260 and the patient, thereby causing positively charged metallic ions to move away from the membrane 260 and toward the blood stream. This erosion may cause unsealing 254 of the outer member 260 and release of the biological agent 246 for treatment upon the targeted tissue, as shown in FIGS. 23C and 23D.

Additionally and/or optionally, the metallic membrane 260 may be coupled with an additional drug or agent. During electrolysis and erosion of the membrane 260, metallic ions carrying the drug or agent may become eroded from membrane 260 and infused into the blood vessel for additional treatment upon die patient.

Alternatively, rather than utilizing metallic materials for outer sheath 260, a thin layer of an electrically sensitive film made from a biodegradable coating can be formed out of bilipid membranes, peptides, and some polyelectrolytes. Such materials may change their structural properties under a DC current, RF energy, or ultrasound energy. These changes may be utilized to trigger the disruptions 254 of the coating film to thus release the drug or agent 246. Moreover, the sensitive film may be additionally and/or alternatively configured to be thermally or pH sensitive as well. Additional films may also include, e.g., proteins such as collagen, fibronectin, albumins, globulins, fibrinogen, and fibrin, with collagen particularly preferred; carboxylated polysaccharides such as polymannuronic acid and polygalacturonic acid; animated polysaccharides, particularly the glycosaminoglycans; e.g., hyaluronic acid; chitin chondroitin sulfate A, B, or C, keratin sulfate, keratosulfate and heparin; and activated polysaccharides such as dextran and starch derivatives.

The applications of the devices and methods discussed above are not limited to the treatments outlined in this application but may include any number of further treatment applications. Modification of the above-described assemblies and methods for carrying out the invention as well as combinations of various features between examples, and variations of aspects of the invention that are obvious to those of skill in the art are intended to be within the scope of this patent.

What is claimed is:

1. An apparatus having a controlled delivery of one or more biologically active substances against or upon a tissue region of interest, comprising:
a catheter having an inflatable balloon;
an outer layer at least partially covering the balloon; and
at least one biologically active substance placed within or upon the outer layer,
wherein expansion of the balloon releases the at least one biologically active substance from the outer layer in a controlled manner for application against or upon the tissue region of interest.

2. The apparatus of claim 1 wherein the catheter comprises an elongate flexible member having the inflatable balloon positioned near or at a distal end of the member.

3. The apparatus of claim 1 wherein the outer layer comprises a material for absorbing and retaining the at least one biologically active substance.

4. The apparatus of claim 3 wherein the material has a compressed state where the biologically active substance is retained within reservoirs which are at least partially closed and an uncompressed state when the balloon is inflated where the biologically active substance is released from opened reservoirs.

5. The apparatus of claim 4 wherein the biologically active substance is released in the uncompressed state when the balloon has an inflated diameter of 1 mm to 10 mm.

6. The apparatus of claim 1 wherein the outer layer comprises a first portion and a second portion such that inflation of the balloon to a first diameter releases a first biologically active substance from the first portion and inflation of the balloon to a second diameter releases the second biologically active substance from the second portion, wherein the second biologically active substance is retained within the second portion until the second diameter is obtained.

7. The apparatus of claim 6 wherein the first diameter ranges from 1 mm to 5 mm and the second diameter ranges from 5 mm to 10 mm.

8. The apparatus of claim 6 wherein the first portion defines a plurality of reservoir having a first reservoir architecture and the second portion defines a plurality of reservoir having a second reservoir architecture different from the first reservoir architecture.

9. The apparatus of claim 8 wherein the first reservoir architecture comprises pores having a size which is smaller than the second reservoir architecture.

10. The apparatus of claim 8 wherein the first reservoir architecture comprises pores having a distribution which is narrower relative to the second reservoir architecture.

11. The apparatus of claim 1 wherein the outer layer is comprised of an elastomeric or non-elastomeric polymer, polyurethane, silicone, pebax, polyimide, polyethylene, polyetheretherketone (PEEK), polyvinylidene fluoride (PVDF) liquid crystal polymer (LCP), polytetrafluoroethylene (PTFE), expanded polytetrafluoroethylene (ePTFE), Hytrel, polyethylene terephthalate (PET), polybutylene terephthalate (PBT) and their copolymers.

12. The apparatus of claim 1 wherein the outer layer is comprised of two or more fiber bundles of polymers.

13. The apparatus of claim 1 wherein the outer layer is comprised of a layered laminate structure having at least a first sheet formed, of first fibers defining a first pore architecture and a second sheet formed of second fibers defining a second pore architecture.

14. The apparatus of claim 13 wherein the first fibers are oriented in a first direction and the second fibers are oriented in a second direction different from the first direction.

15. The apparatus of claim 1 wherein an outer surface of the balloon defines a plurality of reservoirs thereon each configured to expand and release the at least one biologically active substance upon inflation of the balloon.

16. The apparatus of claim 15 wherein the outer surface comprises a sleeve upon which the plurality of reservoirs are defined.

17. The apparatus of claim 15 wherein the plurality of reservoirs are interconnected via channels.

18. The apparatus of claim 15 wherein the plurality of reservoirs are uniformly spaced over the surface of the balloon.

19. The apparatus of claim 15 wherein the plurality of reservoirs comprise a conical or angled configuration.

20. The apparatus of claim 1 wherein the at least one biologically active substance is selected from the group consisting of biopharmaceuticals, anti-infective agents, anti-inflammatory agents, anti-proliferative agents, anti-angiogenic agents, anti-neoplastic agents, anti-scarring agents, scar-inducing agents,, tissue-regenerative agents, anesthetic agents, analgesic agents, immuno-modulating agents, neuro-modulating agents, bioadhesive agents, tissue sealants, and sclerosing agents.

21. The apparatus of claim 1 further comprising a sheath layer disposed at least partially over the outer layer such that the at least one biologically active substance is contained within the outer layer by tire sheath layer.

22. The apparatus of claim 21 wherein the sheath layer is configured to become disrupted upon inflation of the balloon.

23. The apparatus of claim 21 wherein the sheath layer comprises a polymeric biodegradable film configured to dissolve upon, exposure to biological fluids.

24. The apparatus of claim 23 wherein the polymeric biodegradable film is selected from the group consisting of synthetic and naturally occurring polymers, hydrophilic and hydrophobic synthetic polymers, small molecular weight crosslinkers having at least two carbon atoms, proteins, polysaccharides, lipids, DNA and their derivatives, hydrophilic polymers, polyalkylene oxides, polyethylene glycol, poly(ethylene oxide)-poly(propylene oxide) copolymers and their block and random copolymers, glycerol, polyglycerol, highly branched polyglycerol, propyene glycol, trimethylene glycol substituted with one or more polyalkylene oxides, mono-polyoxyethylated glycerol, di-polyoxyethylated glycerol, tri-polyoxyethylated glycerol, mono-polyoxyethylated propylene glycol, di-polyoxyethylated propylene glycol, mono-polyoxyethylated trimetylene glycol, di-polyoxyethylated trimetylene glycol, polyoxyethylated sorbitol, polyoxyethylated glucose, acrylic acid polymers and their analogs and copolymers, polyacrylic acid, polymethacrylic acid, poly(hydroxyethylmethacrylate), poly(hydroxyethylacrylate), poly(methylalkylsulfoxide methacrylate), poly(methylalkylsulfoxide acrylate), aminoethyl acrylate, mono-2-(acryloxy)-ethyl succinate, polymaleic acid, poly(acrylamides), poly(methacrylamide), poly(dimethylacrylamide), and poly(N-isopropyl-acrylamide), poly(olefinic alcohol)s, poly(vinly alcohol), poly(N-vinyl lactams), polyvinyl pyrrolidone), poly(N-vinyl caprolactam), polyoxazonines, poly(methyloxazoline), poly(ethyloxazoline), polyvinylamines, hydrophilic polymers, collagen, fibronectin, albumins, globulins, fibrinogen, fibrin, carboxylated polysaccharides, polymannuronic acid, polygalacturonic acid, aminated polysaccharides, glycosaminoglycans, hyaluronic acid, chitin chondroma sulfate A, B, or C, keratin sulfate, keratosulfate, heparin, activated polysaccharides, dextran, and starch derivatives.

25. The apparatus of claim 21 wherein the sheath layer comprises a metallic erodable membrane configured to erode upon expose to energy.

26. The apparatus of claim 25 further comprising a power supply in electrical communication with the metallic erodable membrane.

27. The apparatus of claim 25 further comprising an additional biologically active substance coupled to the metallic membrane.

28. The apparatus of claim 21 wherein the sheath layer comprises an electrically, thermally, or pH sensitive film selected from the group consisting of bilipid membranes, peptides, poly electrolytes, collagen, fibronectin, albumins, globulins, fibrinogen, fibrin, carboxylated polysaccharides, polymannuronic acid, polygalacturonic acid, aminated polysaccharides, glycosaminoglycans, hyaluronic acid, chitin chondroitin sulfate A, B, or C, keratin sulfate, keratosulfate, heparin, activated polysaccharides, dextran, and starch derivatives.

29. The apparatus of claim 21 wherein the sheath layer is structurally weakened via a plurality of discontinuities along the sheath layer such that inflation of the balloon fragments the sheath layer along the discontinuities.

30. An apparatus having a controlled delivery of one or more biologically active substances against or upon a tissue region of interest, comprising:
a catheter having an inflatable balloon;
a porous outer layer at least partially covering the balloon; and.
at least one biologically active substance placed within or upon the porous outer layer,
wherein expansion of the balloon expands the porous outer layer to release the at least one biologically active substance in a controlled manner for application against or upon the tissue region of interest.

31. The apparatus of claim 30 wherein the catheter comprises an elongate flexible member having the inflatable balloon positioned near or at a distal end of the member.

32. The apparatus of claim 30 wherein the porous outer layer comprises a first portion and a second portion such that inflation of the balloon to a first diameter releases a first biologically active substance from the first portion and inflation of the balloon to a second diameter releases the second biologically active substance from the second portion, wherein the second biologically active substance is retained within the second portion until tire second diameter is obtained.

33. The apparatus of claim 32 wherein the first portion defines a plurality of pores having a first pore architecture and the second portion defines a plurality of pores having a second pore architecture different from the first pore architecture.

34. The apparatus of claim 33 wherein the first pore architecture comprises pores having a size which is smaller than the second pore architecture.

35. The apparatus of claim 33 wherein the first pore architecture comprises pores having a distribution which is narrower relative to the second pore architecture.

36. The apparatus of claim 30 further comprising a sheath layer disposed at least partially over the porous outer layer such that the at least one biologically active substance is contained within the porous outer layer by the sheath layer.

37. The apparatus of claim 36 wherein the sheath layer is configured to become disrupted upon inflation of the balloon.

38. The apparatus of claim 36 wherein the sheath layer comprises a polymeric biodegradable film configured to dissolve upon exposure to biological fluids.

39. The apparatus of claim 38 wherein the polymeric biodegradable film is selected from the group consisting of synthetic and naturally occurring polymers, hydrophilic and hydrophobic synthetic polymers, small molecular weight crosslinkers having at least two carbon atoms, proteins, polysaccharides, lipids, DNA and their derivatives, hydrophilic polymers, polyalkylene oxides, polyethylene glycol, poly(ethylene oxide)-poly(propylene oxide) copolymers and their block and random copolymers, glycerol, polyglycerol, highly branched polyglycerol, propyene glycol, trimethylene glycol substituted with one or more polyalkylene oxides, mono-polyoxyethylated glycerol, di-polyoxyethylated glycerol, tri-polyoxyethylated glycerol, mono-polyoxyethylated propylene glycol, di-polyoxyethylated propylene glycol, mono-polyoxyethylated trimetylene glycol, di-polyoxyethylated trimetylene glycol, polyoxyethylated sorbitol, polyoxyethylated glucose, acrylic acid polymers and their analogs and copolymers, polyacrylic acid, polymethacrylic acid, poly(hydroxyethylmethacrylate), poly(hydroxyethylacrylate), poly(methylalkylsulfoxide methacrylate), poly(methylalkylsulfoxide acrylate), aminoethyl acrylate, mono-2-(acryloxy)-ethyl succinate, polymaleic acid, poly(acrylamides), poly(methacrylamide), poly(dimethylacrylamide), and poly(N-isopropyl-acrylamide), poly(olefinic alcohol)s, poly(vinly alcohol), poly(N-vinyl lactams), poly(vinyl pyrrolidone), poly(N-vinyl caprolactam), polyoxazonines, poly(methyloxazoline), poly(ethyloxazoline), polyvinylamines, hydrophilic polymers, collagen, fibronectin, albumins, globulins, fibrinogen, fibrin, carboxylated polysaccharides, polymannuronic acid, polygalacturonic acid, animated polysaccharides, glycosaminoglycans, hyaluronic acid, chitin chondroitin sulfate A, B, or C, keratin sulfate, keratosulfate, heparin, activated polysaccharides, dextran, and starch derivatives.

40. The apparatus of claim 36 wherein the sheath layer comprises a metallic erodable membrane configured to erode upon expose to energy.

41. The apparatus of claim 40 further comprising a power supply in electrical communication with the metallic erodable membrane.

42. The apparatus of claim 40 further comprising an additional biologically active substance coupled to the metallic membrane.

43. The apparatus of claim 36 wherein the sheath layer comprises an electrically, thermally, or pH sensitive film selected from the group consisting of bilipid membranes, peptides, polyelectrolytes, collagen, fibronectin, albumins, globulins, fibrinogen, fibrin, carboxylated polysaccharides, polymannuronic acid, poly gal acturonic acid, aminated polysaccharides, glycosaminoglycans, hyaluronic acid, chitin chondroitin sulfate A, B, or C, keratin sulfate, keratosulfate, heparin, activated polysaccharides, dextran, and starch derivatives.

44. The apparatus of claim 36 wherein the sheath layer is structurally weakened via a plurality of discontinuities along the sheath layer such that inflation of the balloon fragments the sheath layer along the discontinuities.

45. A method of controlling delivery of one or more biologically active substances against or upon a tissue region of interest, comprising:
positioning a catheter having an inflatable balloon adjacent or proximate to the tissue region;
retaining at least one biologically active substance placed within or upon an outer layer at least partially covering the balloon; and
inflating the balloon such that the outer layer is expanded to release the at least one biologically active substance from the outer layer in a controlled manner for application against or upon the tissue region.

46. The method of claim 45 wherein positioning comprises intravascularly advancing the catheter to the tissue region.

47. The method of claim 45 wherein retaining comprises maintaining the outer layer in a compressed state such that a plurality of reservoirs within the outer layer remain closed to retain the biologically active substance therein.

48. The method of claim 47 wherein inflating further comprises expanding the outer layer into an uncompressed state when the balloon is inflated such that the biologically active substance is released from opened reservoirs.

49. The method of claim 45 wherein inflating comprises expanding the balloon to a diameter of 1 mm to 10 mm.

50. The method of claim 45 further comprising further inflating the balloon to a second larger diameter such that a second biologically active substance is released from the outer layer.

51. The method of claim 45 wherein retaining further comprises retaining the at least one biologically active substance within a plurality of reservoirs each configured to expand and release the biologically active substance upon inflation of the balloon.

52. The method of claim 45 wherein the at least one biologically active substance is selected from the group consisting of biopharmaceuticals, anti-infective agents, anti-inflammatory agents, anti-proliferative agents, anti-angiogenic agents, anti-neoplastic agents, anti-scarring agents, scar-inducing agents, tissue-regenerative agents, anesthetic agents, analgesic agents, immuno-modulating agents, neuro-modulating agents, bioadhesive agents, tissue sealants, and sclerosing agents.

53. The method of claim 45 wherein retaining comprises containing the at least one biologically active substance within the outer layer via a sheath layer disposed at least partially over the outer layer.

54. The method of claim 53 wherein inflating comprises disrupting an integrity of the sheath layer upon inflation of the balloon.

55. The method of claim 53 further comprising dissolving the sheath layer upon exposure to biological fluids.

56. The method of claim 53 further comprising eroding the sheath layer upon exposure to electrical energy.

57. The method of claim 53 further comprising further releasing an additional biologically active substance coupled to the sheath layer.

58. The method of claim 53 wherein inflating the balloon fragments the sheath layer along a plurality of discontinuities along the sheath layer.
